(19) United States
(12) Patent Application Publication
(10) Pub. No.: US 2010/0056426 A1
(43) Pub. Date: Mar. 4, 2010

(54) USE OF IRON CHELATOR FOR THE TREATMENT OF MYOCARDIAL INFARCTION

(75) Inventor: John Glasspool, Basel (CH)

Correspondence Address: NOVARTIS;CORPORATE INTELLECTUAL PROPERTY, ONE HEALTH PLAZA 104/3, EAST HANOVER, NJ, 07936-1080, US

(73) Assignee: NOVARTIS AG, Basel (CH)

(21) Appl. No.: 12/594,979

(22) PCT Filed: May 12, 2008
(86) PCT No.: PCT/EP08/55795

§ 371 Date: Oct. 7, 2009

Related U.S. Application Data
(60) Provisional application No. 60/917,714, filed on May 14, 2007.

(51) Int. Cl. A61K 31/4196; C07D 249/08; A61K 38/28; A61K 31/4965; A61P 9/00

(52) U.S. Cl. 514/4; 548/269.4; 514/255.06; 514/54; 514/383

(57) ABSTRACT

The present invention relates to a method of treating and/or preventing myocardial infarction comprising administering an iron chelator to a warm-blooded animal. The iron chelator is preferably 4-[3,5-bis(2-hydroxyphenyl)-[1,2,4]triazol-1-yl]benzoic.

Iron chelators and their derivatives have been widely-described in the literature. According to the observed binding to iron, the iron chelators may be classified into bidentate, tridentate or hexadentate chelators.

Specific bidentate iron chelators comprise 1,2-dimethyl-3-hydroxypyridin-4-one (Deferiprone, DFP or Ferriprox) and 2-deoxy-2-(N-carbamoylmethyl-[N′-2′-methyl-3′-hydroxypyridin-4′-one])-D-glucopyranose (Feralex-G).

Specific tridentate iron chelators comprise pyridoxal isonicotinyl hydrazone (PIH), 4,5-dihydro-2-(2,4-dihydroxyphenyl)4-methylthiazole-4-carboxylic acid (GT56-252), 4,5-dihydro-2-(3′-hydroxypyridin-2′-yl)4-methylthiazole-4-carboxylic acid (desferrithiocin or DFT) and 4-[3,5-bis(2-hydroxyphenyl)-[1,2,4]triazol-1-yl]benzoic acid (deferasirox). Substituted 3,5-diphenyl-1,2,4-triazoles, e.g. 4-[3,5-bis(2-hydroxyphenyl)-[1,2,4]triazol-1-yl]benzoic acid (deferasirox), their process of manufacture and use thereof are disclosed in the International Patent Publication WO 97149395. A particularly advantageous pharmaceutical preparation of such compounds in the form of dispersible tablets is disclosed in the International Patent Publication WO 2004/035026.

Specific hexadentate iron chelators comprise N,N′-bis(o-hydroxybenzyl)ethylenediamine-N,N′-diacetic acid (HBED), N-(5-C3-L (5-aminopentyl)hydroxycarbamoyl)-propionamido)pentyl)-3(5-(N-hydroxyacetoamido)-pentyl)carbamoyl)-proprionhydroxamic acid (deferoxamine, desferrioxamine or DFO) and hydroxymethyl-starch-bound deferoxamine (S-DFO). Further derivatives of DFO include aliphatic, aromatic, succinic and methylsulphonic analogs of DFO and specifically, sulfonamide-deferoxamine, acetamide-deferoxamine, propylamide deferoxamine, butylamide-deferoxamine, benzoylamide-deferoxamine, succinamide-derferoxamine and methylsulfonamide-deferoxamine.

A further class of iron chelators is the biomimetic class, e.g. as described in Meijler et al., “Synthesis and Evaluation of Iron Chelators with Masked Hydrophilic Moieties” J Amer Chem Soc, 124:1266-1267 (2002), is hereby incorporated by reference in its entirety. These molecules are modified analogues of such naturally produced chelators as DFO and ferrichrome. The analogues allow attachment of lipophilic moieties, e.g. acetoxymethyl ester. The lipophilic moieties are then cleaved intracellularly by endogenous esterases, converting the chelators back into hydrophilic molecules which cannot leak out of the cell.

Various 3,5-diphenyl-1,2,4-triazoles have been known for a long time and their use is described for herbicides, e.g. in EP 185,401, as angiotensin II receptor antagonists in EP 480,659, or very generally as intermediates and starting compounds for fine chemicals, e.g., in JP 06345728.

Certain substituted 3,5-diphenyl-1,2,4-triazoles have valuable pharmacological properties when used in the treatment of disorders which cause an excess of metal in the human or animal body or are caused by it, primarily a marked binding of trivalent metal ions, in particular those of iron [Martell and Motekaitis, Determination and Use of Stability Constants, VCH Publishers, New York (1992)]. They are able, e.g. in an animal model using the non-iron overloaded cholodocostomized rat [Bergeron et al., J Med Chem, 34:2072-2078 (1991)] or the iron-overloaded monkey [Bergeron et al., Blood, 81:2166-2173 (1993)] in doses from approximately 5 μmol/kg, inter alia, to prevent the deposition of iron-containing pigments and in the case of existing iron deposits in the body cause excretion of the iron.

The use of substituted imidazoles as angiotensin II antagonist in the treatment of infarction has been described in the International Patent Publication WO 1992/10180 A1.

However, there is still a need for a treatment for myocardial infarction, both primary and secondary, in mammals.

The present invention provides a method for the treatment and/or prevention of myocardial infarction, e.g. primary or secondary myocardial infarction, comprising administering an amount, e.g. a therapeutically effective amount, of an iron chelator, e.g. a bidentate, tridentate or hexadentate chelators, e.g. 4-[3,5-bis(2-hydroxyphenyl)-[1,2,4]triazol-1-yl]benzoic acid or a salt thereof, to a mammal in need thereof, e.g. a human, e.g. a diabetic patient, an iron overloaded patient, a patient having a disease requiring repeated blood transfusions, or a patient having a combination of at least 2 of those conditions.

In one embodiment of this aspect of the invention the iron chelator is 4-[3,5-bis-(2-hydroxyphenyl)-[1,2,4]-triazol-1-yl]benzoic acid or a salt thereof, or in its free acid form, or its crystalline forms.

4-[3,5-bis-(2-hydroxyphenyl)-[1,2,4]-triazol-1-yl]benzoic acid has the following formula:

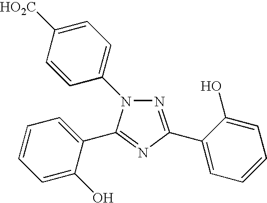

This compound and methods of preparation thereof have been disclosed in U.S. Pat. Nos. 6,465,502 B1 and 6,596,750 B1, the contents of which are incorporated herein in its entirety as if set forth in full herein.

By primary or secondary myocardial infarction is meant the first or second heart attack.

In another aspect of the present invention there is provided a use of an iron chelator, e.g. a bidentate, tridentate or hexadentate chelators, e.g. 4-[3,5-bis-(2-hydroxyphenyl)-[1,2,4]-triazol-1-yl]benzoic acid or a pharmaceutically acceptable salt thereof, for the preparation of a pharmaceutically acceptable medicament for treatment and/or prevention of myocardial infarction, e.g. primary or secondary infarction, e.g. a human, e.g. a diabetic patient, an iron overloaded patient, e.g. a patient having a disease requiring repeated blood transfusions, a patient having a combination of those at least those of those conditions.

The present invention pertain to iron chelators, e.g. a bidentate, tridentate or hexadentate chelators, e.g. 4-[3,5-bis-(2-hydroxyphenyl)-[1,2,4]-triazol-1-yl]benzoic acid or a salt thereof, for the treatment and/or prevention of heart failure or myocardial infarction, e.g. primary or secondary myocardial infarction, in mammals, e.g. a human, e.g. a diabetic patient, an iron overloaded patient, e.g. a patient having a disease requiring repeated blood transfusions, a patient having a combination of those at least those of those conditions.

An aspect of the present invention provides a method for the treatment and/or prevention of heart failure or myocardial infarction, e.g. primary or secondary myocardial infarction, comprising administering an iron chelator, e.g. 4-[3,5-bis-(2-hydroxyphenyl)-[1,2,4]-triazol-1-yl]benzoic acid or a salt thereof, e.g. a therapeutically effective amount of said iron chelator, to a mammal in need thereof, e.g. a human, e.g. in iron overloaded patients with blood disorders requiring repeated blood transfusions.

In a further embodiment of this aspect of the invention the iron chelator is of general formula (I):

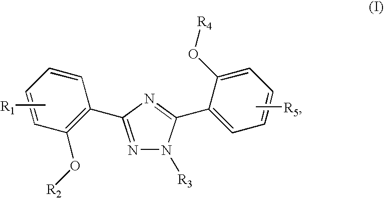

wherein
R₁ and R₅ simultaneously or independently of one another are hydrogen, halogen, hydroxyl, lower alkyl, halo-lower alkyl, lower alkoxy, halo-lower alkoxy, carboxyl, carbamoyl, N-lower alkylcarbamoyl, N,N-di-lower alkylcarbamoyl or nitrile;
R₂ and R₄ simultaneously or independently of one another are hydrogen, unsubstituted or substituted lower alkanoyl or aroyl, or a radical which can be removed under physiological conditions, e.g. a protective group;
R₃ is hydrogen, lower alkyl, hydroxy-lower alkyl, halo-lower alkyl, carboxy-lower alkyl, lower alkoxycarbonyl-lower alkyl, R₆R₇N—C(O)-lower alkyl, unsubstituted or substituted aryl or aryl-lower alkyl, or unsubstituted or substituted heteroaryl or heteroaralkyl; and
R₆ and R₇ simultaneously or independently of one another are hydrogen, lower alkyl, hydroxy-lower alkyl, alkoxy-lower alkyl, hydroxyalkoxy-lower alkyl, amino-lower alkyl, N-lower alkylamino-lower alkyl, N,N-di-lower alkylamino-lower alkyl, N-(hydroxy-lower alkyl)amino-lower alkyl, N,N-di(hydroxy-lower alkyl)amino-lower alkyl, or, together with the nitrogen atom to which they are bonded, form an azaalicyclic ring;
   or a pharmaceutically acceptable salt thereof.

Halogen is, e.g. chlorine, bromine or fluorine, but can also be iodine.

The prefix “lower” designates a radical having not more than 7 and in particular not more than 4 carbon atoms.

Alkyl is straight-chain or branched. Per se, e.g. lower alkyl, or as a constituent of other groups, e.g. lower alkoxy, lower alkylamine, lower alkanoyl, lower alkylaminocarbonyl, it can be unsubstituted or substituted, for example by halogen, hydroxyl, lower alkoxy, trifluoromethyl, cyclo-lower alkyl, azaalicyclyl or phenyl, it is preferably unsubstituted or substituted by hydroxyl.

Lower alkyl is, e.g. n-propyl, isopropyl, n-butyl, isobutyl, sec-butyl, tert-butyl, n-pentyl, neopentyl, n-hexyl or n-heptyl, preferably methyl, ethyl and n-propyl. Halo-lower alkyl is lower alkyl substituted by halogen, preferably chlorine or fluorine, in particular, by up to three chlorine or fluorine atoms.

Lower alkoxy is, e.g. n-propoxy, isopropoxy, n-butoxy, isobutoxy, sec-butoxy, tert-butoxy, n-amyloxy, isoamyloxy, preferably methoxy and ethoxy. Halo-lower alkoxy is lower alkoxy substituted by halogen, preferably chlorine or fluorine, in particular, by up to three chlorine or fluorine atoms.

Carbamoyl is the radical H₂N—C(O)—, N-lower alkylcarbamoyl is lower alkyl-HN—C(O)— and N,N-di-lower alkylcarbamoyl is di-lower alkyl-N—C(O)—.

Lower alkanoyl is HC(O)— and lower alkyl-C(O)— and is, e.g. acetyl, propanoyl, butanoyl or pivaloyl.

Lower alkoxycarbonyl designates the radical lower alkyl-O—C(O)— and is, e.g. n-propoxycarbonyl, isopropoxycarbonyl, n-butoxycarbonyl, isobutoxycarbonyl, sec-butoxycarbonyl, tert-butoxycarbonyl, n-amyloxycarbonyl, isoamyloxycarbonyl, preferably methoxycarbonyl and ethoxycarbonyl.

Aryl, per se, e.g., aryl, or as a constituent of other groups, e.g., aryl-lower alkyl or aroyl is, e.g., phenyl or naphthyl, which is substituted or unsubstituted. Aryl is preferably phenyl which is unsubstituted or substituted by one or more, in particular, one or two, substituents, e.g. lower alkyl, lower alkoxy, hydroxyl, nitro, halogen, trifluoromethyl, carboxyl, lower alkoxycarbonyl, amino, N-lower alkylamino, N,N-di-lower alkylamino, aminocarbonyl, lower alkylaminocarbonyl, di-lower alkylaminocarbonyl, heterocycloalkyl, heteroaryl or cyano. Primarily, aryl is unsubstituted phenyl or naphthyl, or phenyl which is substituted by lower alkyl, lower alkoxy, hydroxyl, halogen, carboxyl, lower alkoxycarbonyl, N,N-di-lower alkylamino or heterocycloalkylcarbonyl.

Aroyl is the radical aryl-C(O)— and is, e.g., benzoyl, toluoyl, naphthoyl or p-methoxybenzoyl.

Aryl-lower alkyl is, e.g., benzyl, p-chlorobenzyl, o-fluorobenzyl, phenylethyl, p-tolylmethyl, p-dimethylaminobenzyl, p-diethylaminobenzyl, p-cyanobenzyl, p-pyrrolidinobenzyl.

Heterocycloalkyl designates a cycloalkyl radical having 3-8, in particular, having from 5 to not more than 7, ring atoms, of which at least one is a heteroatom; oxygen, nitrogen and sulfur are preferred. Azaalicyclyl is a saturated cycloalkyl radical having 3-8, in particular, 5-7, ring atoms, in which at least one of the ring atoms is a nitrogen atom. Azaalicyclyl can also contain further ring heteroatoms, e.g., oxygen, nitrogen or sulfur; it is, e.g., piperidinyl, piperazinyl, morpholinyl or pyrrolidinyl. Azaalicyclyl radicals can be unsubstituted or substituted by halogen or lower alkyl. The azaalicyclyl radicals bonded via a ring nitrogen atom, which are preferred, are, as is known, designated as piperidino, piperazino, morpholino, pyrrolidino etc.

Heteroaryl per se, e.g., heteroaryl, or as a constituent of other substituents, e.g., heteroaryl-lower alkyl, is an aromatic radical having from 3 to not more than 7, in particular, from 5 to not more than 7, ring atoms, in which at least one of the ring atoms is a heteroatom, e.g., pyrrolyl, imidazolyl, triazolyl, tetrazolyl, oxazolyl, thiazolyl, furanyl, thiophenyl, pyridyl, pyrazinyl, oxazinyl, thiazinyl, pyranyl or pyrimidinyl. Heteroaryl can be substituted or unsubstituted. Heteroaryl which is unsubstituted or substituted by one or more, in particular one or two, substituents, e.g., lower alkyl, halogen, trifluoromethyl, carboxyl or lower alkoxycarbonyl, is preferred.

Heteroaryl-lower alkyl designates a lower alkyl radical in which at least one of the hydrogen atoms, preferably on the terminal C atom, is replaced by a heteroaryl group if the alkyl chain contains two or more carbon atoms.

N-lower alkylamino is, e.g. n-propylamino, n-butylamino, i-propylamino, i-butylamino, hydroxyethylamino, preferably methylamino and ethylamino. In N,N-di-lower alkylamino, the alkyl substituents can be identical or different. Thus, N,N-di-lower alkylamino is, e.g. N,N-dimethylamino, N,N-diethylamino, N,N-methylethylamino, N-methyl-N-morpholinoethylamino, N-methyl-N-hydroxyethylamino or N-methyl-N-benzylamino.

Protective groups, their introduction and removal are described, e.g. in McOmie, Protective Groups in Organic Chemistry, Plenum Press, London, New York (1973), and in Methoden der organischen Chemie [Methods of organic chemistry], Houben-Weyl, 4th Edition, Vol. 1571, Georg Thieme, Stuttgart (1974), and also in Greene, Protective Groups in Organic Synthesis, John Wiley, New York (1981). It is characteristic of protective groups that they can be removed easily, i.e. without undesired side reactions taking place, e.g. solvolytically, reductively, photolytically or alternatively under physiological conditions.

Hydroxyl groups can be present, e.g. in the form of an easily cleavable ester or ether group, preferably of an alkanoyl or aralkanoyl ester group or of a cycloheteroalkyl, aralkyl or alkoxyalkyl ether group, but also of a silyl ester or silyl ether group, in particular, as an acetyl or benzoyl ester or as a tetrahydropyranyl, benzyl or methoxymethyl ether.

Salts of compounds of the formula (I) are pharmaceutically acceptable salts, especially salts with bases, such as appropriate alkali metal or alkaline earth metal salts, e.g., sodium, potassium or magnesium salts, pharmaceutically acceptable transition metal salts, such as zinc salts, or salts with organic amines, such as cyclic amines, such as mono-, di- or tri-lower alkylamines, such as hydroxy-lower alkylamines, e.g. mono-, di- or trihydroxy-lower alkylamines, hydroxy-lower alkyl-lower alkylamines or polyhydroxy-lower alkylamines. Cyclic amines are, e.g. morpholine, thiomorpholine, piperidine or pyrrolidine. Suitable mono-lower alkylamines are, e.g. ethyl- and tert-butylamine; di-lower alkylamines are, e.g., diethyl- and diisopropylamine; and tri-lower alkylamines are, e.g. trimethyl- and triethylamine. Appropriate hydroxy-lower alkylamines are, e.g. mono-, di- and triethanolamine; hydroxy-lower alkyl-lower alkylamines are, e.g. N,N-dimethylamino- and N,N-diethylaminoethanol; a suitable polyhydroxy-lower alkylamine is, e.g. glucosamine. In other cases it is also possible to form acid addition salts, e.g. with strong inorganic acids, such as mineral acids, e.g., sulfuric acid, a phosphoric acid or a hydrohalic acid, with strong organic carboxylic acids, such as lower alkanecarboxylic acids, e.g. acetic acid, such as saturated or unsaturated dicarboxylic acids, e.g. malonic, maleic or fumaric acid or, such as hydroxycarboxylic acids, e.g. tartaric or citric acid, or with sulfonic acids, such as lower alkane- or substituted or unsubstituted benzenesulfonic acids, e.g. methane- or p-toluenesulfonic acid. Compounds of the formula (I) having an acidic group, e.g. carboxyl, and a basic group, e.g. amino, can also be present in the form of internal salts, i.e. in zwitterionic form, or a part of the molecule can be present as an internal salt, and another part as a normal salt.

The compounds, including their salts, can also be in the form of hydrates or solvates, or their crystals can include, e.g. the solvent used for crystallization.

The compounds of formula (I) and their salts, depending on the choice of the starting substances and working procedures, can be present in the form of one of the possible isomers, e.g., stereo-isomers or tautomers, or as a mixture thereof. In this context, pure isomers obtainable are, e.g., pure enantiomers, pure diastereoisomers or pure tautomers. Correspondingly, isomer mixtures which can be present are, e.g. racemates or diastereoisomer mixtures. Isomer mixtures of compounds of formula (I), in free form or in salt form, can be separated into the components in a customary manner, e.g. on the basis of the physicochemical differences of the constituents, in a known manner by fractional crystallization, distillation and/or chromatography. Advantageously, the more active isomer is isolated.

4-[3,5-bis-(2-hydroxyphenyl)-[1,2,4]-triazol-1-yl]benzoic acid is an iron chelator that has been shown to be effective in the selective removal of iron in model systems and in humans. See, e.g., Hershko et al., Blood, 97:1115-1122 (2001); and Nisbet Brown et al., Lancet, 361:1597-1602 (2003).

In one embodiment of this aspect of the invention, the myocardial infarction is a primary or secondary myocardial infarction and the patients being treated are diabetic patients.

In another aspect of the present invention there is provided the use of an iron chelator for the treatment and/or prevention of myocardial infarction.

The compounds of the invention can also be used in formulations together with other active agents, in particular, those used for the treatment of diabetes and hypertension, e.g.

In the therapeutic use for primary and secondary prevention of infarction, the compounds of formula (I) are incorporated into standard pharmaceutical compositions. They can be administered orally, parenterally, rectally, topically or transdermally.

Suitable pharmaceutical preparations of a compound of formula (I) are those for enteral, in particular, oral, and furthermore rectal, administration, and those for parenteral administration to warm-blooded animals, especially to man, the pharmacological active ingredient being present on its own or together with customary pharmaceutical adjuncts. The pharmaceutical preparations contain (in percentages by weight), e.g., from approximately 0.001-100%, preferably from approximately 0.1% to approximately 100%, more preferably from approximately 0.1% to approximately 50%, of the active ingredient.

Oral formulations of 4-[3,5-bis(2-hydroxyphenyl)-[1,2,4]triazol-1-yl]benzoic acid or a pharmaceutically acceptable salt thereof are disclosed in the following International Patent Application publication WO 2004/035026, the contents of which are incorporated herein by reference in their entirety as if set forth in full herein.

Pharmaceutical preparations for enteral or parenteral administration are, e.g. those in unit dose forms, such as sugar-coated tablets, tablets, dispersible tablets, effervescent tablets, capsules, suspendable powders, suspensions or suppositories or ampoules. These are prepared in a manner known per se, e.g., by means of conventional pan-coating, mixing, granulation or lyophilization processes. Pharmaceutical preparations for oral administration can thus be obtained by combining the active ingredient with solid carriers, if desired granulating a mixture obtained and processing the mixture or granules, if desired or necessary, after addition of suitable adjuncts to give tablets or sugar-coated tablet cores.

Suitable carriers are, in particular, fillers, such as sugars, e.g., lactose, sucrose, mannitol or sorbitol, cellulose preparations and/or calcium phosphates, e.g., tricalcium phosphate or calcium hydrogen phosphate, furthermore, binders, such as starch pastes, using, e.g., maize, wheat, rice or potato starch, gelatin, tragacanth, methylcellulose and/or polyvinylpyrrolidone, and, if desired, disintegrants, such as the abovementioned starches, furthermore carboxymethyl starch, crosslinked polyvinylpyrrolidone, agar or alginic acid or a salt thereof, such as sodium alginate. Adjuncts are primarily flow-regulating and lubricating agents, e.g., salicylic acid, talc, stearic acid or salts thereof, such as magnesium or calcium stearate, and/or polyethylene glycol. Sugar-coated tablet cores are provided with suitable, if desired enteric, coatings, using, inter alia, concentrated sugar solutions which, if desired, contain gum arabic, talc, polyvinylpyrrolidone, polyethylene glycol and/or titanium dioxide, coating solutions in suitable organic solvents or solvent mixtures or, for the preparation of enteric coatings, solutions of suitable cellulose preparations, such as acetylcellulose phthalate or hydroxypropylmethylcellulose phthalate. Colorants or pigments, e.g., for the identification or the marking of various doses of active ingredient, can be added to the tablets or sugar-coated tablet coatings.

Dispersible tablets are tablets which rapidly disintegrate in a comparatively small amount of liquid, e.g., water, and which, if desired, contain flavorings or substances for masking the taste of the active ingredient. They can advantageously be employed for the oral administration of large individual doses, in which the amount of active ingredient to be administered is so large that on administration as a tablet which is to be swallowed in undivided form or without chewing that it can no longer be conveniently ingested, in particular, by children. Further orally administrable pharmaceutical preparations are hard gelatin capsules and also soft, closed capsules of gelatin and a plasticizer, such as glycerol or sorbitol. The hard gelatin capsules can contain the active ingredient in the form of granules, e.g., as a mixture with fillers, such as lactose, binders, such as starches, and/or glidants, such as talc or magnesium stearate, and, if desired, stabilizers. In soft capsules, the active ingredient is preferably dissolved or suspended in suitable liquids, such as fatty oils, liquid paraffin or liquid polyethylene glycols, it also being possible to add stabilizers.

Moreover, suspendable powders, e.g. those which are described as “powder in bottle”, abbreviated “PIB”, or ready-to-drink suspensions, are suitable for an oral administration form. For this form, the active ingredient is mixed, e.g. with pharmaceutically acceptable surface-active substances, e.g. sodium lauryl sulfate or polysorbate, suspending auxiliaries, e.g. hydroxypropylcellulose, hydroxypropylmethylcellulose or another known from the prior art and previously described, e.g. in “Handbook of Pharmaceutical Excipients”, pH regulators, such as citric or tartaric acid and their salts or a USP buffer and, if desired, fillers, e.g. lactose, and further auxiliaries, and dispensed into suitable vessels, advantageously single-dose bottles or ampoules. Immediately before use, a specific amount of water is added and the suspension is prepared by shaking. Alternatively, the water can also be added even before dispensing.

Rectally administrable pharmaceutical preparations are, e.g. suppositories which consist of a combination of the active ingredient with a suppository base. A suitable suppository base is, e.g. natural or synthetic triglycerides, paraffin hydrocarbons, polyethylene glycols or higher alkanols. Gelatin rectal capsules can also be used which contain a combination of the active ingredient with a base substance. Possible base substances are, e.g. liquid triglycerides, polyethylene glycols or paraffin hydrocarbons.

For parenteral administration, aqueous solutions of an active ingredient in water-soluble form, e.g. of a water-soluble salt, are primarily suitable; furthermore suspensions of the active ingredient, such as appropriate oily injection suspensions, suitable lipophilic solvents or vehicles, such as fatty oils, e.g. sesame oil, or synthetic fatty acid esters, e.g. ethyl oleate or triglycerides, being used, or aqueous injection suspensions which contain viscosity-increasing substances, e.g. sodium carboxymethylcellulose, sorbitol and/or dextran, and, if desired, also stabilizers.

The dosage of the active ingredient, in particular of a compound of formula (I), can depend on various factors, such as activity and duration of action of the active ingredient, severity of the illness to be treated or its symptoms, manner of administration, warm-blooded animal species, sex, age, weight and/or individual condition of the warm-blooded animal. The doses to be administered daily in the case of oral administration are between 10 mg/kg and approximately 120 mg/kg, in particular, 20 mg/kg and approximately 80 mg/kg, and for a warm-blooded animal having a body weight of approximately 40 kg, preferably between approximately 400 mg and approximately 4,800 mg, in particular approximately 800 3,200 mg, which is expediently divided into 2-12 individual doses.

The present invention also pertains to a pharmaceutical composition containing 4-[3,5-bis(2-hydroxyphenyl)-[1,2,4]triazol-1-yl]benzoic acid as defined herein, either alone or in a combination with another therapeutic agent, e.g. each at an effective therapeutic dose as reported in the art, said therapeutic agent is selected from the group of:
a) anti-diabetic agent such as insulin, insulin derivatives and mimetics; insulin secretagogues such as the sulfonylureas, e.g. Glipizide, glyburide and Amaryl; insulinotropic sulfonylurea receptor ligands such as meglitinides, e.g. nateglinide and repaglinide; peroxisome proliferator-activated receptor (PPAR) ligands; protein tyrosine phosphatase-1B (PTP-1B) inhibitors such as PTP-112; GSK3 (glycogen synthase kinase-3) inhibitors such as SB-517955, SB-4195052, SB-216763, NN-57-05441 and NN-57-05445; RXR ligands such as GW-0791 and AGN-194204; sodium-dependent glucose cotransporter inhibitors such as T-1095; glycogen phosphorylase A inhibitors such as BAY R3401; biguanides such as metformin; alpha-glucosidase inhibitors such as acarbose; GLP-1 (glucagon like peptide-1), GLP-1 analogs such as Exendin-4 and GLP-1 mimetics; and DPPIV (dipeptidyl peptidase IV) inhibitors such as LAF237;
b) hypolipidemic agent such as 3-hydroxy-3-methyl-glutaryl coenzyme A (HMG-CoA) reductase inhibitors, e.g. lovastatin, pitavastatin, simvastatin, pravastatin, cerivastatin, meva-statin, velostatin, fluvastatin, dalvastatin, atorvastatin, rosuvastatin and rivastatin; squalene synthase inhibitors; FXR (farnesoid X receptor) and LXR (liver X receptor) ligands; cholestyramine; fibrates; nicotinic acid and aspirin;
c) anti-obesity agent such as orlistat; and
d) anti-hypertensive agent, e.g. loop diuretics such as ethacrynic acid, furosemide and torsemide; angiotensin converting enzyme (ACE) inhibitors such as benazepril, captopril, enalapril, fosinopril, lisinopril, moexipril, perinodopril, quinapril, ramipril and trandolapril; inhibitors of the Na—K-ATPase membrane pump such as digoxin; neutralendopeptidase (NEP) inhibitors; ACE/NEP inhibitors such as omapatrilat, sampatrilat and fasidotril; angiotensin II antagonists such as candesartan, eprosartan, irbesartan, losartan, telmisartan and valsartan, in particular valsartan; β-adrenergic receptor blockers such as acebutolol, atenolol, betaxolol, bisoprolol, metoprolol, nadolol, propranolol, sotalol and timolol; inotropic agents such as digoxin, dobutamine and milrinone; calcium channel blockers such as amLodipine, bepridil, diltiazem, felodipine, nicardipine, nimodipine, nifedipine, nisoldipine and verapamil; aldosterone receptor antagonists; and aldosterone synthase inhibitors.

Other specific anti-diabetic compounds are described by Patel Mona in Expert Opin Investig Drugs, 2003, 12(4), 623-633, in the FIGS. 1 to 7, which are herein incorporated by reference. A compound of the present invention may be administered either simultaneously, before or after the other active ingredient, either separately by the same or different route of administration or together in the same pharmaceutical formulation.

The following examples are intended to illustrate the invention described above, but without restricting it to them.

### EXAMPLE 1

### Pharmaceutical Preparations

### REFERENCE EXAMPLES A TO D

### As Disclosed in U.S. Pat. No. 6,465,504 B1

The expression “active ingredient” is below to be understood as meaning a compound of the formula (I), in free form or in the form of a pharmaceutically acceptable salt, in particular, a compound of the type which is described as a product in one of the above examples.

### EXAMPLE A

Tablets, comprising 200 mg of active ingredient each, can be prepared, e.g., as follows:

Composition (for 10,000 tablets):

| | |
|---|---|
| active ingredient | 2000.0 g |
| lactose | 500.0 g |
| potato starch | 352.0 g |
| gelatin | 8.0 g |
| talc | 60.0 g |
| magnesium stearate | 10.0 g |
| silica (highly disperse) | 20.0 g |
| ethanol | q.s. |

The active ingredient is mixed with the lactose and 292 g of potato starch, and the mixture is moistened with an ethanolic solution of the gelatin and granulated through a sieve. After drying, the remainder of the potato starch, the magnesium stearate, the talc and the silica is admixed and the mixture is compressed to give tablets of weight 295.0 mg each and 200 mg active ingredient content, which, if desired, can be provided with breaking notches for finer adjustment of the dosage.

### EXAMPLE B

Coated tablets, each comprising 400 mg of active ingredient, can be prepared, e.g., as follows:

Composition (for 1,000 tablets):

| | |
|---|---|
| active ingredient | 400.0 g |
| lactose | 100.0 g |
| maize starch | 70.0 g |
| talc | 8.5 g |
| calcium stearate | 1.5 g |
| hydroxypropylmethylcellulose | 2.36 g |
| shellac | 0.64 g |
| water | q.s. |
| dichloromethane | q.s. |

The active ingredient, the lactose and 40 g of the maize starch are mixed and moistened and granulated with a paste prepared from 15 g of maize starch and water (with warming). The granules are dried, and the remainder of the maize starch, the talc and the calcium stearate is added and mixed with the granules. The mixture is compressed to give tablets and these are coated with a solution of hydroxypropylmethylcellulose and shellac in dichloromethane; final weight of the coated tablet: 583 mg.

### EXAMPLE C

Hard gelatin capsules, comprising 500 mg of active ingredient, can be prepared, e.g., in the following manner:

Composition (for 1.000 capsules):

| | |
|---|---|
| active ingredient | 500.0 g |
| lactose | 250.0 g |
| microcrystalline cellulose | 30.0 g |
| sodium lauryl sulfate | 2.0 g |
| magnesium stearate | 8.0 g |

The sodium lauryl sulfate is sieved into the lyophilized active ingredient through a sieve having a mesh width of 0.2 mm. Both components are intimately mixed. Then the lactose is first sieved in through a sieve having a mesh width of 0.6 mm and the microcrystalline cellulose is then sieved in through a sieve having a mesh width of 0.9 mm. After that, the ingredients are again intimately mixed for 10 minutes. Finally, the magnesium stearate is sieved in through a sieve having a mesh width of 0.8 mm. After 3 minutes' further mixing, 790 mg each of the formulation obtained are dispensed into hard gelatin capsules of suitable size.

### EXAMPLE D

Oral suspension powder, comprising 300 mg of active ingredient, can be prepared, e.g., as follows;

Composition (1 administration):

| | |
|---|---|
| active ingredient | 300 mg |
| hydroxypropylcellulose (Klucel HF) | 50 mg |
| tartaric acid | 100 mg |
| sodium lauryl sulfate | 100 mg |

The sodium lauryl sulfate is sieved into the lyophilized active ingredient through a sieve having a mesh width of 0.2 mm. Both components are intimately mixed. Then the microcrystalline cellulose is sieved in through a sieve having a mesh width of 0.9 mm. After this, the ingredients are again intimately mixed for 10 minutes. Finally, the tartaric acid is sieved in through a sieve having a mesh width of 0.8 mm. After 3 minutes' further mixing, the mixture is dispensed into a container having a capacity of at least 10 mL. For use, the mixture is made up to 10 mL with water and vigorously shaken.

### EXAMPLE 2

### Biological Tests

Compounds of formula (I) and their pharmaceutically acceptable salts have pharmacological activity and are useful as pharmaceuticals, for the prevention of primary and secondary prevention of infarction, as may be demonstrated in animal test methods, e.g., in accordance with the following test method:

Myocardial infarction (MI) is induced in anesthetized, diabetic and non-diabetic male Sprague-Dawley rats via occlusion of the left coronary artery. Animals are initially anesthetized either with sodium pentobarbital (40-50 mg/kg, intraperitoneal injection) or isoflurane (continuous inhalation). Avertin (0.2-0.4 mg/g) is used prior to intubation of the animal when isoflorane is used as anesthetic. The level of anesthesia is monitored by assessing the pedal or tail pinch reflex. The anesthetized animal is then artificially ventilated (Model 683, Harvard Apparatus) and the chest cavity opened by an incision at the left fourth intercostal space. The heart is exposed, pericardial sac opened and separated, and left anterior descending (LAD) coronary artery exposed. Occlusion of the LAD is effected by ligation with a 6-0 silk suture passed with a tapered needle underneath the LAD about 3-4 mm below the tip of the left auricle. Occlusion is confirmed by development of pallor of the anterior wall of the left ventricle. A drop of 1% lidocaine is placed on the apex of the heart to prevent arrhythmias. Lungs are inflated and the chest cavity, muscles and skin are closed layer by layer with 4-0 nylon and 4-0 absorbable (for muscles) sutures. The wound is treated with betadine and the animals are allowed to recover from anesthesia. Test drugs are administered orally or parenterally (subcutaneous, intraperitoneal, intravenous). Blood samples are periodically withdrawn for assessment of drug exposure levels. Echocardiographic measurements are performed before randomization (to ensure homogenous distribution among the various study groups) and periodically during the course of the study. For this purpose, rats are anesthetized with 2-3% isoflurane, the left hemithorax shaved and pre-warmed ultrasound transmission gel applied to the precordium. Animals are then placed on a heating pad, and transthoracic echocardiography is performed using GE Vivid 7 echocardiographic machine equipped with a 15-MHz linear transducer. Rat hearts are imaged at the papillary muscle level and wall thickness and chamber dimensions are measured from a midventricular, short-axis view. Septal and posterior end-diastolic and end-systolic wall thicknesses and left ventricular internal dimensions are measured according to the American Society of Echocardiography leading-edge method. From the right lateral decubitus position, apical four-chamber view is obtained, mitral inflow velocities recorded with pulsed-wave Doppler, and early mitral acceleration time and deceleration time measured. All images are stored in digital format and analyzed subsequently. For more detailed and precise analysis, select animals are subjected to magnetic resonance imaging assessments.

Hemodynamic assessment are performed in rats to obtain functional correlates of the structural data previously obtained. Blood pressure—volume measurements are ascertained via the right carotid artery. In these procedures, the carotid artery is dissected from the vagal nerve and two silk sutures are placed underneath it. The distal suture is tied off at the level of bifurcation of the common carotid artery and the proximal suture is tied into the loose loop about 5 mm away. A small clamp is placed on the most proximal portion of the artery to stop blood flow. A small incision is made in the artery with a 20-gauge needle bent at the very tip. A 2.0 French (smaller size for smaller rats) Millar pressure-volume catheter is moved below the needle into the artery and stabilized by lightly tightening of the proximal suture. The clamp is released and the catheter is gently moved forward 10-12 mm into the aortic arch to measure the systemic blood pressure. Pressure signals are recorded at 2 kHz for 2 minutes, and then the catheter is moved another 10 mm further down to pass through the aortic valve and enter the left ventricular chamber. Ventricular pressure-volume tracings are recorded for another 30-50 minutes, stored to disk, and analyzed using PowerLab software (Chart 5, ADInstruments). At termination of the study, animals are euthanized and select organs (heart, lungs and liver) excised, weighed and saved for RNA analysis and histology.

Hyperglycemia is induced in the rats by administering a single intraperitoneal injection of streptozotocin. To ensure that the animals are diabetic, urine analysis is performed 24 hours later using Chemstrip uGK (Roche Diagnostics, Indianapolis, Ind.). Rats with urine glucose values of >2000 mg/dL with polyuria 24 hours after STZ injection are considered to be diabetic. Rats with urine glucose values of <2000 mg/dL after 24 hours are considered to be non-diabetic and are excluded from further study. Two weeks after induction of diabetes, diabetic animals are subjected to coronary artery ligation to induce MI as described above.

Administration of iron chelators either prior to or after induction of MI surprisingly affords beneficial effects. When administered prior to induction of MI, it significantly decreases infarct size and inhibits adverse remodeling of the heart. The animals exhibit significantly improved cardiac function and inhibition of progression to heart failure relative to vehicle controls. These effects are also associated with significant improvements in survival. The improvements in cardiac function and survival of the animals are also observed in diabetic MI animals. Surprisingly, the iron chelators also inhibit adverse cardiac remodeling when administered post MI to either non-diabetic or diabetic animals. These effects are also associated with significant survival benefits.

### EXAMPLE 3

### Pharmaceutical Preparations: Dispersible Tablet Formulation (125 mg, 250 mg and 500 mg dispersible Tablets) with a Disintegration Time Below 3 Minutes

| | | | Amount per dispersible tablet | | |
|---|---|---|---|---|---|
| | | | (mg) | | |
| Components | | % | 125 mg | 250 mg | 500 mg |
| Phase I | Compound I (free acid form) | 29.4 | 125.0 | 250.0 | 500.0 |
| | Lactose 200 Mesh (1.1) | 17.1 | 72.6 | 145.2 | 290.4 |
| | Crospovidone XL (1.2) | 15.0 | 63.7 | 127.4 | 254.8 |
| Phase II | PVP K.30 (1.3) | 3.0 | 12.8 | 25.6 | 51.2 |
| | Sodium laurylsulfate (1.4) | 0.5 | 2.1 | 4.2 | 8.4 |
| Phase III | Crospovidone XL (1.2) | 5.0 | 21.3 | 42.6 | 85.2 |
| | Microcrystalline cellulose (1.1) | 14.9 | 63.3 | 126.6 | 253.2 |
| | Lactose spray dried (1.1) | 14.9 | 63.3 | 126.6 | 253.2 |
| | Aerosil 200 (1.5) | 0.2 | 0.9 | 1.8 | 3.6 |
| Phase IV | Magnesium stearate (1.6) | <0.2* | | | |

| | | | | |
|---|---|---|---|---|
| Tablet weight (mg) | 100.0 | 425 | 850 | 1700 |
| Tablet diameter (mm) | — | 12 | 15 | 20 |
| Tablet thickness (mm) | — | 3.6 +/− 0.3 | 4.5 +/− 0.3 | 5.5 +/− 0.3 |

Dispersible tablets of Compound I free acid according to the invention are prepared by forming a inner phase by wet granulation of a mixture of Phase I and Phase II ingredients, Phase III ingredients formed the outer phase and the lubricant (Phase IV) is sprayed directly onto the punches of the tabletting machine. * 0.1% w/w of magnesium stearate is equivalent to 1000 ppm.

1. An iron chelator for use in the treatment and/or prevention of myocardial infarction comprising administering a therapeutically effective amount of said iron chelator to a mammal in need thereof.

2. An iron chelator according to claim 1, wherein the iron chelator is a bidentate, tridentate or hexadentate iron chelator.

3. An iron chelator according to claim 1, in which the iron chelator is a compound of the formula (I): 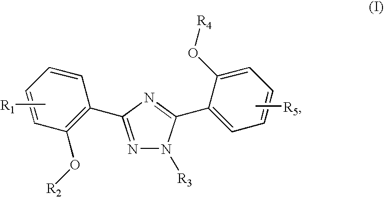
wherein
R₁ and R₅ simultaneously or independently of one another are hydrogen, halogen, hydroxyl, lower alkyl, halo-lower alkyl, lower alkoxy, halo-lower alkoxy, carboxyl, carbamoyl, N-lower alkylcarbamoyl, N,N-di-lower alkylcarbamoyl or nitrile;
R₂ and R₄ simultaneously or independently of one another are hydrogen, unsubstituted or substituted lower alkanoyl or aroyl, or a radical which can be removed under physiological conditions, e.g., a protective group;
R₃ is hydrogen, lower alkyl, hydroxy-lower alkyl, halo-lower alkyl, carboxy-lower alkyl, lower alkoxycarbonyl-lower alkyl, R₆R₇N—C(O)-lower alkyl, unsubstituted or substituted aryl or aryl-lower alkyl, or unsubstituted or substituted heteroaryl or heteroaralkyl;
R₆ and R₇ simultaneously or independently of one another are hydrogen, lower alkyl, hydroxy-lower alkyl, alkoxy-lower alkyl, hydroxyalkoxy-lower alkyl, amino-lower alkyl, N-lower alkylamino-lower alkyl, N,N-di-lower alkylamino-lower alkyl, N-(hydroxy-lower alkyl)amino-lower alkyl, N,N-di(hydroxy-lower alkyl)amino-lower alkyl or, together with the nitrogen atom to which they are bonded, form an azaalicyclic ring;
and pharmaceutically acceptable salts thereof.

4. (canceled)

5. A iron chelator according to claim 1 wherein the myocardial infarction is a primary or secondary myocardial infarction.

6. An iron chelator of claim 1, wherein the mammal is a diabetic patient or a patient being iron overloaded, or a patient with blood disorders requiring repeated blood transfusions, or a patient having a combination of those indications.

7. A pharmaceutical formulation comprising an iron chelator and an anti-diabetic agent selected from the group consisting of insulin, insulin derivatives and mimetics, Glipizide, glyburide, Amraryl, nateglinide, repaglinide, peroxisome proliferator-activated receptor (PPAR) ligands, PTP-112, SB-517955, SB-4195052, SB-216763, NN-57-05441, NN-57-05445, GW-0791, AGN-194204, T-1095, BAY R3401, metforming, acarbose, and LAF237.

8. A pharmaceutical formulation comprising an iron chelator and an anti-hypertensive active ingredient selected from the group consisting of ethacrynic acid, furosemide, torsemide, benazepril, captopril, enalapril, fosinopril, lisinopril, moexipril, perinodopril, quinapril, ramipril, trandolapril, digoxin, neutralendopeptidase (NEP) inhibitors, omapatrilat, sampatrilat, fasidotril, candesartan, eprosartan, irbesartan, losartan, telmisartan, valsartan, acebutolol, atenolol, betaxolol, bisoprolol, metoprolol, nadolol, propranolol, sotalol, timolol, digoxin, dobutamine, milrinone, amLodipine, bepridil, diltiazem, felodipine, nicardipine, nimodipine, nifedipine, nisoldipine, verapamil, aldosterone receptor antagonists and aldosterone synthase inhibitors.

9. An iron chelator according to claim 1, or a pharmaceutical formulation according to claim wherein the iron chelator is 4-[3,5-bis(2-hydroxyphenyl)-[1,2,4]triazol-1-yl]benzoic acid or a pharmaceutically acceptable salt thereof.

10. A pharmaceutical formulation according to claim 7 for the treatment and/or prevention of myocardial infarction comprising administering a therapeutically effective amount of said pharmaceutical formulation to a mammal in need thereof.

11. A pharmaceutical formulation according to claim 7 for the treatment of a mammal wherein the mammal is a diabetic patient or a patient being iron overloaded, or a patient with blood disorders requiring repeated blood transfusions, or a patient having a combination of those indications.

12. A pharmaceutical formulation according to claim 8 for the treatment and/or prevention of myocardial infarction comprising administering a therapeutically effective amount of said pharmaceutical formulation to a mammal in need thereof.

13. A pharmaceutical formulation according to claim 8 for the treatment of a mammal wherein the mammal is a diabetic patient or a patient being iron overloaded, or a patient with blood disorders requiring repeated blood transfusions, or a patient having a combination of those indications.
